(19) United States
(12) Patent Application Publication
(10) Pub. No.: US 2024/0226142 A9
(43) Pub. Date: Jul. 11, 2024

(54) SODIUM BICARBONATE INJECTABLE FORMULATION AND METHODS THEREOF

(71) Applicant: Endo Ventures Limited, Dublin (IE)

(72) Inventors: Kumaresh SOPPIMATH, Skillman, NJ (US); Tushar HINGORANI, Bridgewater, NJ (US)

(21) Appl. No.: 18/278,153

(22) PCT Filed: Feb. 25, 2022
(86) PCT No.: PCT/US2022/018003

§ 371 Date: Aug. 21, 2023

Related U.S. Application Data
(60) Provisional application No. 63/154,625, filed on Feb. 26, 2021.

(51) Int. Cl. A61K 33/10; A61J 1/14; A61K 9/00; A61K 33/00

(52) U.S. Cl. CPC A61K 33/10; A61J 1/1468; A61K 9/0019; A61K 33/00

(57) ABSTRACT

The present disclosure is directed to ready-to-use, injectable formulations of a liquid pharmaceutical composition comprising sodium bicarbonate, and methods of treatment using said formulations.

### FIELD OF THE INVENTION

The present invention relates to injectable, ready-to-use liquid pharmaceutical compositions of sodium bicarbonate. The present invention also relates to methods of treating diseases or disorders characterized by metabolic acidosis or a loss of sodium bicarbonate.

### BACKGROUND OF THE INVENTION

Normal pH of blood is slightly basic, maintained at around 7.4. When the pH of blood plasma becomes too acidic, dropping below the normal range of about 7.35 to 7.45, a subject may experience, depending on the degree of acidity in the blood, decreasing levels of consciousness, including coma, and disruption of multiple organ systems, such as cardiac arrest. Sodium bicarbonate is commonly used as a systemic alkalizing agent to correct for an acid-base imbalance in medical conditions characterized by metabolic acidosis or a loss of sodium bicarbonate.

Metabolic acidosis arises from an accumulation of anions in relative excess to cations in the blood plasma, which reduces blood pH. Metabolic acidosis can be caused by diabetic ketoacidosis, lactic acidosis, septic shock, or chronic kidney dysfunction that produces an excess net dietary acid load. In addition, metabolic acidosis can also occur intraoperatively or due to cardiac arrest. Although successful long-term management of metabolic acidosis requires therapy of the underlying disorder, sodium bicarbonate is often used to alleviate the acute symptoms associated with metabolic acidosis and its underlying causative disorders.

Sodium bicarbonate replacement therapy can be used to treat acute or chronic loss of sodium bicarbonate, for instance in subjects experiencing hyperchloremic acidosis as a result of diarrhea or renal proximal tubular acidosis, rather than acid buildup or retention in the blood. Sodium bicarbonate is also the primary buffer used in dialysis fluids for subjects with renal dysfunction. Hemodialysis is usually performed in a hospital or specialized clinic, under controlled conditions using complex equipment. Peritoneal dialysis, although a simpler procedure that can be performed outside of a hospital or clinic setting, necessitates frequent administration of accurate concentrations of reagents under sterile conditions each time. As it is often impractical or cumbersome to mix dialysis solutions at the site of administration, shelf-stable premixed formulations are preferred.

Sodium bicarbonate is typically administered as an intravenous injection or infusion to treat metabolic acidosis occurring due to poor tissue perfusion or renal failure. Administration of sodium bicarbonate can delay or eliminate the need for further dialysis, alone or in combination with continuous renal replacement therapy (CRRT). Treatment using sodium bicarbonate primarily takes place in an operating room, emergency room, oncology unit, or during resuscitation in the critical care unit of a hospital or medical facility. Pharmaceutical formulations of sodium bicarbonate currently available on the market include ampules, vials, and prefilled syringes that contain sodium bicarbonate in the form of either a dry powder or a liquid concentrate.

Intravenous infusion of sodium bicarbonate currently requires on-site complex manipulation by hospital pharmacy staff of existing powder or concentrated formulations of sodium bicarbonate to produce an appropriate final concentration for infusion. Preparation of the final concentration is time-consuming and often requires combining multiple vials of sodium bicarbonate into one intravenous (IV) bag. Moreover, compounding multiple IV bags of sodium bicarbonate can be challenging, requiring high capital and human resource costs. Multiple hospital pharmacy staff members must work together to prepare multiple sterile IV bags of sodium bicarbonate in strict compliance with USP 797 and ASHP formulation guidelines. Each preparation involves the potential for contamination, waste, and medication errors. Additional difficulty in the management of inventory and third-party suppliers create a further burden on the administration of sodium bicarbonate.

Thus, there is a need for a premixed, aseptic, and shelf-stable liquid pharmaceutical formulation of sodium bicarbonate that is ready-to-use for intravenous injection or infusion in a hospital or clinical setting, without complex on-site manipulation of the formulation.

### BRIEF SUMMARY OF THE INVENTION

The present disclosure provides liquid pharmaceutical compositions comprising sodium bicarbonate and one or more pharmaceutically acceptable excipients.

In some aspects, the liquid pharmaceutical composition comprising sodium bicarbonate is aseptic, premixed, and ready-to-use.

In some aspects, the liquid pharmaceutical composition comprising sodium bicarbonate is packaged in a container for intravenous use.

In some aspects, the container for intravenous use is packaged in an overwrap.

In some aspects, the pH of the liquid pharmaceutical composition increases by less than 0.5 after storage for at least 6 months at 25° C.

In some aspects, the sodium bicarbonate is present in a concentration of from about 0.001 mEq/mL to about 10 mEq/mL.

In some aspects, the sodium bicarbonate is present in a concentration of from about 0.01 mEq/mL to about 1 mEq/mL.

In some aspects, the sodium bicarbonate is present in a concentration of from about 0.05 mEq/mL to about 0.50 mEq/mL.

In some aspects, the sodium bicarbonate is present in a concentration of from about 0.10 mEq/mL to about 0.20 mEq/mL.

In some aspects, the sodium bicarbonate is present in a concentration of about 0.10 mEq/mL.

In some aspects, the sodium bicarbonate is present in a concentration of about 0.15 mEq/mL.

In some aspects, the sodium bicarbonate is present in a concentration of about 0.20 mEq/mL.

In some aspects, the sodium bicarbonate is present in a concentration of about 0.3 mEq/mL.

In some aspects, the sodium bicarbonate is present in a concentration of about 1 mEq/mL.

In some aspects, the pH of the composition is from about 7.5 to about 8.5.

In some aspects, the pH of the composition is from about 7.7 to about 8.5.

In some aspects, the pH of the composition is from about 8 to about 8.5.

In some aspects, the sodium bicarbonate is mixed in a total volume of about 250 mL of diluent.

In some aspects, the sodium bicarbonate is mixed in a total volume of about 500 mL of diluent.

In some aspects, the composition is isotonic.

In some aspects, the composition is hypertonic.

In some aspects, the composition is hypotonic.

In some aspects, the composition comprises a tonicity agent.

In some aspects, the tonicity agent is dextrose.

In some aspects, the composition is stable for at least 6 months at 25° C.

In some aspects, the composition is stable for at least 12 months at 25° C.

In some aspects, the composition is stable for at least 18 months at 25° C.

In some aspects, the composition does not contain a preservative.

In some aspects, the composition is packaged under nitrogen atmosphere.

In some aspects, the composition is packaged under carbon dioxide atmosphere.

In some aspects, carbon dioxide is present inside the container at a concentration of less than about 5%.

In some aspects, the composition is substantially free of impurities.

In some aspects, the container for intravenous use is an infusion bag.

In some aspects, the container for intravenous use comprises a material through which carbon dioxide is impermeable.

In some aspects, the material through which carbon dioxide is impermeable comprises silicon oxide (SiOₓ).

In some aspects, the container for intravenous use comprises polypropylene, SiOₓ, titanium oxide, ethylene vinyl acetate (EVA), polyvinyl chloride (PVC), or combinations thereof.

In some aspects, the container for intravenous use comprises an inner material and an outer material.

In some aspects, the inner material and outer material are selected from polypropylene, SiOₓ, titanium oxide, EVA, PVC, or combinations thereof.

In some aspects, the inner material and outer material are the same.

In some aspects, the inner material and outer material are the same.

In some aspects, the inner material and outer material comprise silicon oxide (SiOₓ).

In some aspects, the inner material and outer material are different In some aspects, at least one of the inner material and outer material comprises silicon oxide (SiOₓ).

In some aspects, the overwrap comprises aluminum, polypropylene, SiOₓ, titanium oxide, EVA, PVC, or combinations thereof.

In some aspects, the overwrap comprises an inner material and an outer material.

In some aspects, the inner material and outer material are selected from aluminum, polypropylene, SiOₓ, titanium oxide, EVA, PVC, or combinations thereof.

In some aspects, the inner material and outer material are the same.

In some aspects, the inner material and outer material are the same and comprises silicon oxide (SiOₓ).

In some aspects, the inner material and outer material are different.

In some aspects, the inner material and outer material are different.

In some aspects, at least one of the inner material and outer material comprises silicon oxide (SiOₓ).

In some aspects, the space between the container and the overwrap is evacuated.

In some aspects, the space between the container and the overwrap is filled with nitrogen.

In some aspects, the space between the container and the overwrap is filled with carbon dioxide.

In some aspects, the container for intravenous use is a single-dose container.

In some aspects, the premixed liquid is sterilized without heat.

The present disclosure also provides methods for alleviating metabolic acidosis in a subject, the method comprising administering to the subject an aseptic, ready-to-use, premixed liquid pharmaceutical composition comprising sodium bicarbonate and one or more pharmaceutically acceptable excipients, wherein the composition is administered to a subject having an arterial blood pH of <7.5.

In some aspects, the metabolic acidosis is due to diabetic ketoacidosis.

In some aspects, the metabolic acidosis is due to lactic acidosis.

In some aspects, the metabolic acidosis is due to septic shock.

In some aspects, the metabolic acidosis is due to intraoperative metabolic acidosis.

In some aspects, the metabolic acidosis is due to cardiac arrest.

In some aspects, the composition is sterile and aseptic upon administration to a subject having an arterial blood pH of <7.5.

The present disclosure also provides methods of replacing sodium bicarbonate in a subject with sodium bicarbonate loss, the method comprising administering to the subject an aseptic, ready-to-use, premixed liquid pharmaceutical composition comprising sodium bicarbonate and one or more pharmaceutically acceptable excipients.

In some aspects, the sodium bicarbonate loss is due to hyperchloremic acidosis.

In some aspects, the hyperchloremic acidosis is due to diarrhea or renal proximal tubular acidosis.

In some aspects, the composition is sterile and aseptic upon administration to the subject.

The present disclosure also provides methods of treating kidney dysfunction in a subject, the method comprising administering to the subject an aseptic, ready-to-use, premixed liquid pharmaceutical composition comprising sodium bicarbonate and one or more pharmaceutically acceptable excipients.

### BRIEF DESCRIPTION OF THE DRAWINGS

FIG. 1 is a bar graph showing the change in pH over time of sodium bicarbonate solutions packaged in a polypropylene bag (manufacturer: Technoflex) or a SiOₓ bag (manufacturer: Technoflex or Polycine).

FIG. 2 is a bar graph showing the change in pH over time of sodium bicarbonate solutions packaged in IV bags at concentrations of 0.15 mEq/mL, 0.3 mEq/mL, or 1 mEq/mL.

FIG. 3 is a bar graph showing the change in pH over time of sodium bicarbonate solutions packaged in IV bags with or without an overpouch and/or evacuating the space between the IV bag and the overpouch.

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. These materials, methods, and examples are intended only to be illustrative, and not intended to be limiting. Other features and advantages of the present invention will be apparent to a person of ordinary skill in the art from reading the detailed description and the claims.

To further define the present disclosure, the following terms and definitions are provided.

The singular forms “a,” “an,” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

“Administration” or “administer” means giving or providing a pharmaceutical composition to a subject. The pharmaceutical compositions disclosed herein can be administered “intravenously,” that is administered by direct insertion of a needle into the vein of a subject to deliver to the subject a solution containing a reagent or drug given in a single dose or by continuous infusion. For example, to alleviate metabolic acidosis, intravenous infusion of a liquid pharmaceutical composition comprising sodium carbonate contained in an IV bag can be performed to administer said composition to a subject.

The term “pharmaceutically acceptable” as used herein refers to compounds, materials, compositions, formulations, and/or dosage forms which are, within the scope of sound medical judgment, suitable for use in contact with the tissues of human beings and animals without excessive toxicity, irritation, allergic response, or any other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition. The excipient can be an inert substance, an inactive substance, and/or a not medicinally active substance.

The term “diluent” refers to any combination of pharmaceutically acceptable fluids, including only water, or a mixture of water and a buffer, preservative, stabilizer, lubricant, tonicity agent, sugar, or sugar alcohol. Pharmaceutical compositions of the present disclosure comprise one or more pharmaceutically active agents premixed with the diluent prior to packaging, transport, and/or administration to a subject. The diluent is sterile upon premixing with the one or more pharmaceutically active agents of the present disclosure.

The term “aseptic” as used herein refers to compounds, materials, compositions, agents, or solutions that are made free from contamination caused by bacteria, viruses, or other living microorganisms that are not desired to be contained therein. Once made, aseptic compounds, materials, compositions, agents, and solutions will not create or reproduce any kind of undesired living microorganism therein. These compounds, materials, compositions, agents, and solutions shall remain aseptic throughout the manufacturing process, during transportation and storage, and/or at the point of administration to a subject.

The term “sterile” as used herein refers to the absence or complete elimination of all microorganisms from compounds, materials, compositions, agents, or solutions, as a result of physical or chemical processes used to destroy all living microorganisms therein, including bacteria, viruses, and fungi. The compounds, materials, compositions, agents, and solutions shall remain sterile throughout the manufacturing process, during transportation and storage, and/or at the point of administration to a subject.

The term “ready-to-use” as used herein refers to pharmaceutical compounds, materials, compositions, agents, or solutions that can be administered to a subject without a need, prior to administration thereof in a subject, for additional processing steps, such as dilution or compounding of the container containing the pharmaceutical compound, material, composition, agent, or solution.

The terms “effective amount” or “pharmaceutically effective amount” or “therapeutically effective amount” where used herein refer to the amount or quantity of a drug or pharmaceutically active substance which is sufficient to elicit the required or desired therapeutic response, or in other words, the amount which is sufficient to elicit an appreciable biological response when administered to a subject.

The term “substantially free” refers to a composition that is at least about 95% pure by weight, at least about 98% pure by weight, or at least about 99% pure by weight, and contains less than about 5% by weight, less than about 2% by weight, or less than about 1% by weight of contaminants. In other words, only trace amounts of contaminants such as reference substances not having a substantial effect on the composition, impurity compounds, metabolites, or degradation products, can be detected.

As used herein, “treat,” “treatment,” “treating,” “alleviate,” and “alleviating” refer to the reduction or amelioration of the progression, severity, and/or duration of a given disease, disorder, or condition, as a result of the administration of one or more therapies disclosed herein (including, but not limited to, a liquid pharmaceutical composition comprising sodium carbonate).

The term “mEq/mL” as used herein refers to the concentration of a solution as measured by the ratio between the number of particles of a solute, generally an active pharmaceutical ingredient or excipient, that will react with a certain number of hydrogen ions when dissolved in a solvent, generally but not necessarily water, and the amount of solvent. The number of solute particles dispersed in the solvent depends on the valence of the solute. The unit of milliequivalents (mEq) is represented by the following equation:

$$mEq = \frac{( mass ) ( valence )}{molecular weight}$$

For example, a 10 mEq/mL sodium bicarbonate solution represents a solution comprising 10 mEq of sodium bicarbonate for every 1 mL water.

The terms “mOsm/mL” or “osmolarity” refer to the concentration of a solution as measured by the ratio between the number of osmoles of solute per liter (may be expressed in mL) of solution. For example, the osmolarity of a 1 mEq solution of sodium bicarbonate equals 2.0 mOsm/mL. Body fluids are usually maintained within the range of 0.28 to 0.30 mOsm/mL.

### 2. Pharmaceutical Compositions

Pharmaceutical compositions of the present disclosure include an effective amount of a liquid pharmaceutical composition comprising sodium bicarbonate and one or more pharmaceutically acceptable excipients. In some aspects, the liquid pharmaceutical composition comprising sodium bicarbonate is aseptic. In some aspects, the liquid pharmaceutical composition comprising sodium bicarbonate is sterile. In some aspects, the liquid pharmaceutical composition comprising sodium bicarbonate is premixed. In some aspects, the liquid pharmaceutical composition comprising sodium bicarbonate is ready-to-use. In some aspects, the liquid pharmaceutical composition comprising sodium bicarbonate is ready-to-use, premixed, aseptic and/or sterile. In some aspects, sodium bicarbonate is the sole therapeutically effective agent in the liquid pharmaceutical composition.

In some aspects, the sodium bicarbonate is present in the liquid pharmaceutical composition at a concentration of from about 0.001 mEq/mL to about 10 mEq/mL. In some aspects, the sodium bicarbonate is present in a concentration of from about 0.01 mEq/mL to about 1 mEq/mL, from about 0.05 mEq/mL to about 0.50 mEq/mL, or from about 0.10 mEq/mL to about 0.20 mEq/mL. In some aspects, the sodium bicarbonate is present in a concentration of about 0.10 mEq/mL, about 0.11 mEq/mL, about 0.12 mEq/mL, about 0.13 mEq/mL, about 0.14 mEq/mL, about 0.15 mEq/mL, about 0.16 mEq/mL, about 0.17 mEq/mL, about 0.18 mEq/mL, about 0.19 mEq/mL, or about 0.20 mEq/mL. In some aspects, the sodium bicarbonate is present in a concentration of about 0.15 mEq/mL. In some aspects, the sodium bicarbonate is present in a concentration of about 0.3 mEq/mL, about 0.4 mEq/mL, about 0.5 mEq/mL, about 0.6 mEq/mL, about 0.7 mEq/mL, about 0.8 mEq/mL, about 0.9 mEq/mL, about 1 mEq/mL, about 1.25 mEq/mL, or about 1.5 mEq/mL. The referenced concentrations of sodium bicarbonate solution can be readily converted from mEq/mL to mOsm/mL.

In some aspects, the sodium bicarbonate of the liquid pharmaceutical composition is mixed in a total volume of diluent. In some aspects, the sodium bicarbonate is mixed in a total volume of from about 100 mL of diluent to about 2000 mL of diluent. In some aspects, the sodium bicarbonate is mixed in a total volume of from about 200 mL of diluent to about 1000 mL of diluent. In some aspects, the sodium bicarbonate is mixed in a total volume of about 200 mL, about 250 mL, about 300 mL, about 350 mL, about 400 mL, about 450 mL, about 500 mL, about 550 mL, about 600 mL, about 650 mL, about 700 mL, about 750 mL, about 800 mL, about 850 mL, about 900 mL, about 950 mL, or about 1000 mL of diluent. In some aspects, the sodium bicarbonate is mixed in a total volume of about 250 mL of diluent. In some aspects, the sodium bicarbonate is mixed in a total volume of about 500 mL of diluent.

In some aspects, the liquid pharmaceutical composition is isotonic. In some aspects, the liquid pharmaceutical composition is hypertonic. In some aspects, the liquid pharmaceutical composition is hypotonic.

In some aspects, the liquid pharmaceutical composition comprises a tonicity agent. Examples of tonicity agents include, but are not limited to, salts, particularly sodium chloride, potassium chloride, glycerin, mannitol, dextrose, and other sugar alcohols, and other suitable pharmaceutically acceptable tonicity regulators and mixtures thereof. In some aspects, the tonicity agent is dextrose.

In some aspects, the pH of the liquid pharmaceutical composition is about 8. In some aspects, the pH of the liquid pharmaceutical composition is about 7.5, about 7.6, about 7.7, about 7.8, about 7.9, about 8.1, about 8.2, about 8.3, about 8.4, about 8.5, about 8.6, about 8.7, about 8.8, about 8.9, or about 9. In some aspects, the pH of the liquid pharmaceutical composition is from about 7.5 to about 7.7, from about 7.5 to about 7.9, from about 7.5 to about 8, from about 7.5 to about 8.2, from about 7.5 to about 8.4, from about 7.5 to about 8.5, from about 7.5 to about 8.7, from about 7.5 to about 9, from about 7.7 to about 7.9, from about 7.7 to about 8, from about 7.7 to about 8.2, from about 7.7 to about 8.4, from about 7.7 to about 8.5, from about 7.7 to about 8.7, from about 7.7 to about 9, from about 7.9 to about 8, from about 7.9 to about 8.2, from about 7.9 to about 8.4, from about 7.9 to about 8.5, from about 7.9 to about 8.7, from about 7.9 to about 9, from about 8 to about 8.2, from about 8 to about 8.4, from about 8 to about 8.5, from about 8 to about 8.7, from about 8 to about 9, from about 8.2 to about 8.4, from about 8.2 to about 8.5, from about 8.2 to about 8.7, from about 8.2 to about 9, from about 8.4 to about 8.5, from about 8.4 to about 8.7, from about 8.4 to about 9, from about 8.5 to about 8.7, from about 8.5 to about 9, or from about 8.7 to about 8.9.

In some aspects, the liquid pharmaceutical composition is shelf-stable for at least 6 months at 25° C. or at room temperature. In some aspects, the liquid pharmaceutical composition is shelf-stable for at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months, at 25° C. or at room temperature. In some aspects, the liquid pharmaceutical composition is shelf-stable for at least 12 months at 25° C. or at room temperature. In some aspects, the liquid pharmaceutical composition is shelf-stable for at least 12 months at 25° C. or at room temperature. In some aspects, the liquid pharmaceutical composition is shelf-stable for at least 18 months at 25° C. or at room temperature. In some aspects, the liquid pharmaceutical composition is shelf-stable for at least 24 months at 25° C. or at room temperature.

In some aspects, the liquid pharmaceutical composition is packaged in a container for intravenous use. In some aspects, the container for intravenous use is an infusion bag, a semi-rigid plastic infusion container, a glass infusion container, or a prefilled syringe or other injection device. In some aspects, the container for intravenous use comprises a material through which carbon dioxide is impermeable. In some aspects, the material through which carbon dioxide is impermeable comprises silicon oxide (SiOₓ). In some aspects, the container for intravenous use comprises polypropylene, SiOₓ, titanium oxide, ethylene vinyl acetate (EVA), polyvinyl chloride (PVC), polyolefin, cyclic olefin copolymer, polycarbonate, polyethylene, high density polyethylene, metal foil covered plastic, polymethacrylate ether, polyacrylic acid, polyamide, polyimide, polysulfone, polyester, or a combination thereof. In some aspects, the container for intravenous use comprises an inner material and an outer material. In some aspects, the inner material and outer material are selected from polypropylene, SiOₓ, titanium oxide, ethylene vinyl acetate (EVA), polyvinyl chloride (PVC), polyolefin, cyclic olefin copolymer, polycarbonate, polyethylene, high density polyethylene, metal foil covered plastic, polymethacrylate ether, polyacrylic acid, polyamide, polyimide, polysulfone, polyester, or a combination thereof. In some aspects, the inner material and outer material are the same. In some aspects, the inner material and outer material are different. In other aspects, the container for intravenous use is a single-dose container. In some aspects, the packaging of the liquid pharmaceutical composition in the container for intravenous use is performed under sterile and/or aseptic conditions. In some aspects, the premixed liquid of the liquid pharmaceutical composition is sterilized without heat. In some aspects, the liquid pharmaceutical composition in the container for intravenous use is sterile and/or aseptic upon administration to a subject.

In some aspects, the container for intravenous use is packaged in an overwrap. In some aspects, the overwrap comprises aluminum, polypropylene, SiOₓ, titanium oxide, EVA, PVC, polyolefin, cyclic olefin copolymer, polycarbonate, polyethylene, high density polyethylene, metal foil covered plastic, or a combination thereof. In some aspects, the overwrap comprises an inner material and an outer material. In some aspects, the inner material and outer material are selected from aluminum, polypropylene, SiOₓ, titanium oxide, EVA, PVC, polyolefin, cyclic olefin copolymer, polycarbonate, polyethylene, high density polyethylene, metal foil covered plastic, or a combination thereof. In some aspects, the inner material and the outer material are the same. In some aspects, the inner material and the outer material are different. In some aspects, the space between the container and the overwrap is evacuated. In some aspects, the space between the container and the overwrap is filled with carbon dioxide, nitrogen, or combinations thereof.

In some aspects, the liquid pharmaceutical composition is substantially free of contaminants, including impurities, metabolites, or degradation products, or any combination thereof. In some aspects, the liquid pharmaceutical composition is packaged under nitrogen atmosphere. In other aspects, the liquid pharmaceutical composition is packaged under carbon dioxide atmosphere. In some aspects, the liquid pharmaceutical composition is packaged under nitrogen and carbon dioxide atmosphere. In some aspects, carbon dioxide is used to adjust and maintain the pH of the liquid pharmaceutical composition inside the container for intravenous use. In some aspects, carbon dioxide is present inside the container at a concentration of less than about 5%, less than about 4%, less than about 3%, less than about 2%, or less than about 1% of the total concentration of the liquid pharmaceutical composition inside the container.

In some aspects, the liquid pharmaceutical composition comprises one or more pharmaceutically acceptable excipients. In some aspects, the excipient is water, a preservative, a buffer, a stabilizer, a viscosity agent, a tonicity regulator, a chelating agent, a polymer, a lipid, or any combination thereof. The one or more excipients can serve various purposes. A person skilled in the art can select one or more excipients with respect to the particular desired properties by routine experimentation and without any undue burden. The amount of each excipient used can vary within conventional ranges known in the art. Techniques and excipients that can be used to formulate pharmaceutical compositions are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21^{st} edition, Gennaro, Ed., Lippincott Williams & Wilkins (2005).

In some aspects, the liquid pharmaceutical composition comprises a preservative. Non-limiting examples of preservatives include benzoates, such as sodium benzoate, sorbic acid, sorbates, such as potassium sorbate, benzalkonium chloride, ethylenediaminetetraacetic acid (EDTA), methyl and ethyl parabens, chlorhexidine, hexetidine, vitamin E, and any combination thereof. The amount of preservative in the liquid pharmaceutical composition is a concentration effective to preserve the composition.

In some aspects, the liquid pharmaceutical composition does not contain a preservative.

In some aspects, the liquid pharmaceutical composition comprises one or more antimicrobial agents. Non-limiting examples of antimicrobial agents include gentamycin, tobramycin, paromomycin, kanamycin, neomycin, vancomycin, amikacin, moxifloxacin, gatifloxacin, levofloxacin, gemifloxacin, ciprofloxacin, norfloxacin, ofloxacin, sulfacetamide, sulfadiazine, sulfadimidine, sulfafurazole, sulfisomidine, sulfadoxine, sulfamethoxazole, sulfamoxole, sulfanitran, sulfadimethoxine, sulfamethoxypyridazine, sulfametoxydiazine, sulfametopyrazine, penicillin G, penicillin V, benzylpenicillins, phenoxymethylpenicillin, dicloxacillin, flucloxacillin, oxacillin, nafcillin, amoxicillin, ticarcillin, piperacillin, ritipenem, ertapenem, doripenem, imipenem and meropenem, cefazolin, cefalexin, cefadroxil, cefapirin, cefaclor, cefotetan, cephamycin, cefprozil, cefuroxime axetil, ceftriaxone, ceftazidime, cefoperazone, cefdinir, cefcapene, cefdaloxime, ceftizoxime, cefmenoxime, cefotaxime, cefpiramide, cefpodoxime, ceftibuten, cefditoren, cefepime, ceftaroline fosamil, ceftolozane, ceftobiprole, ceftiofur, cefquinome and cefovecin, aztreonam, sulbactam, tazobactam, clavulanic acid, avibactam, or cyclosporine, or any combination thereof

### 3. Methods of Treatment

The present disclosure also provides a method for alleviating metabolic acidosis in a subject, the method comprising administering to the subject a therapeutically effective amount of a liquid pharmaceutical composition comprising sodium bicarbonate and one or more pharmaceutically acceptable excipients. In some aspects, the liquid pharmaceutical composition is aseptic. In some aspects, the liquid pharmaceutical composition is sterile. In some aspects, the liquid pharmaceutical composition is premixed. In some aspects, the liquid pharmaceutical composition is ready-to-use. In some aspects, the liquid pharmaceutical composition is ready-to-use, premixed, aseptic and/or sterile. In some aspects, the liquid pharmaceutical composition is sterile and aseptic upon administration to the subject.

In some aspects, the method for alleviating metabolic acidosis in a subject comprises administering the liquid pharmaceutical composition to a subject having an arterial blood pH of less than about 7.5. In some aspects, the method for alleviating metabolic acidosis comprises administering the liquid pharmaceutical composition to a subject having an arterial blood pH of less than about 7.4, less than about 7.3, less than about 7.2, less than about 7.1, or less than about 7.0. In some aspects, the method for alleviating metabolic acidosis comprises administering the liquid pharmaceutical composition to a subject having an arterial blood pH of <7.5. In some aspects, the method for alleviating metabolic acidosis comprises administering the liquid pharmaceutical composition to a subject having an arterial blood pH of <7.4. In some aspects, the method for alleviating metabolic acidosis comprises administering the liquid pharmaceutical composition to a subject having an arterial blood pH of <7.3.

In some aspects, the metabolic acidosis is due to diabetic ketoacidosis. In some aspects, the metabolic acidosis is due to lactic acidosis. In some aspects, the metabolic acidosis is due to septic shock. In some aspects, the metabolic acidosis is due to chronic kidney dysfunction that produces excess net dietary acid load. In some aspects, the metabolic acidosis is due to intraoperative metabolic acidosis, which is an acute condition often caused by poor tissue perfusion during a surgical operation. In some aspects, the metabolic acidosis is due to cardiac arrest, which is an acute condition often caused by poor tissue perfusion and increased lactate production.

The present disclosure also provides a method of replacing sodium bicarbonate in a subject with sodium bicarbonate loss, the method comprising administering to the subject a therapeutically effective amount of a liquid pharmaceutical composition comprising sodium bicarbonate and one or more pharmaceutically acceptable excipients. In some aspects, the liquid pharmaceutical composition is aseptic. In some aspects, the liquid pharmaceutical composition is sterile. In some aspects, the liquid pharmaceutical composition is premixed. In some aspects, the liquid pharmaceutical composition is ready-to-use. In some aspects, the liquid pharmaceutical composition is ready-to-use, premixed, aseptic and/or sterile. In some aspects, the liquid pharmaceutical composition is sterile and aseptic upon administration to the subject.

In some aspects, the sodium bicarbonate loss is due to hyperchloremic acidosis. In some aspects, the hyperchloremic acidosis is due to diarrhea. In some aspects, the hyperchloremic acidosis is due to renal proximal tubular acidosis.

The present disclosure also provides a method of treating kidney dysfunction in a subject, the method comprising administering to the subject a therapeutically effective amount of a liquid pharmaceutical composition comprising sodium bicarbonate and one or more pharmaceutically acceptable excipients. In some aspects, the liquid pharmaceutical composition is aseptic. In some aspects, the liquid pharmaceutical composition is sterile. In some aspects, the liquid pharmaceutical composition is premixed. In some aspects, the liquid pharmaceutical composition is ready-to-use. In some aspects, the liquid pharmaceutical composition is ready-to-use, premixed, aseptic and/or sterile. In some aspects, the liquid pharmaceutical composition is sterile and aseptic upon administration to the subject.

### EXAMPLES

### Examples 1-6

Table 1 provides details of six exemplary intravenous formulations of a liquid pharmaceutical composition comprising sodium bicarbonate (Examples 1-6) of the present disclosure.

TABLE 1

| | Example | Example | Example | Example | Example | Example |
|---|---|---|---|---|---|---|
| | 1 | 2 | 3 | 4 | 5 | 6 |
| Sodium | 0.1 | 0.25 | 0.5 | 0.75 | 0.8 | 1 |
| Bicarbonate(mEq/mL) | | | | | | |
| Carbon Dioxide | q.s. pH | q.s. pH | q.s. pH | q.s. pH | q.s. pH | q.s. pH |
| | 7.0 to 8.5 | 7.0 to 8.5 | 7.0 to 8.5 | 7.0 to 8.5 | 7.0 to 8.5 | 7.0 to 8.5 |
| Nitrogen | Process | Process | Process | Process | Process | Process |
| | aid | aid | aid | aid | aid | aid |

### Example 7

Effect of Bag Type on Solution pH

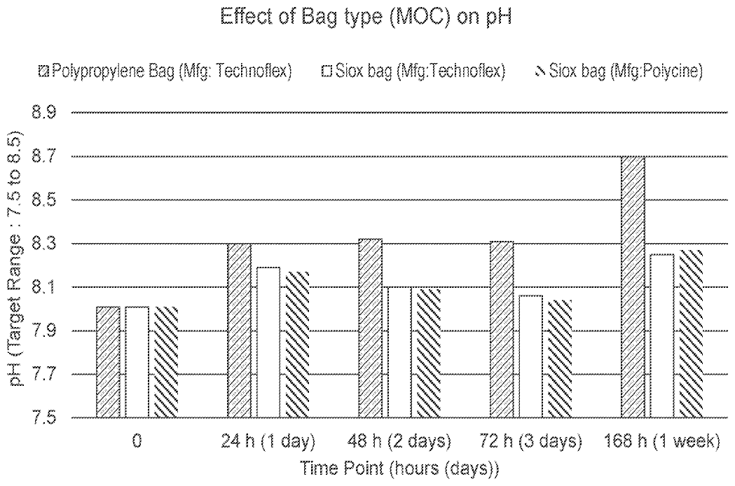

The effect of IV bag type on the pH of sodium bicarbonate solutions was tested. Sodium bicarbonate solutions were added to a polypropylene bag manufactured by Technoflex, a polypropylene bag with inner SiOₓ layer manufactured by Technoflex, and a polypropylene bag with inner SiOₓ layer manufactured by Polycine. The pH of the solution in each bag was measured at 0 days, 1 day, 2 days, 3 days, and 7 days. The results are shown in FIG. 1. The results surprisingly show that the pH of the solutions stored in bags comprising an inner SiOₓ layer increased less over time compared to the pH of solution stored in a polypropylene bag.

### Example 8

Effect of Concentration on Solution pH

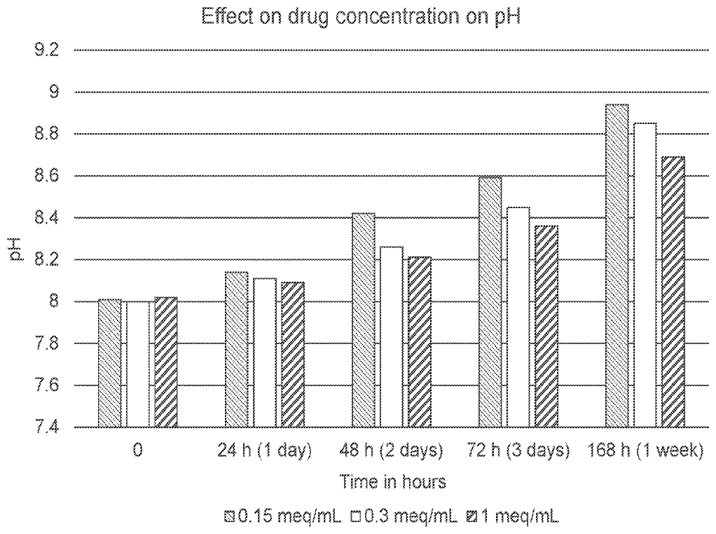

The effect of sodium bicarbonate concentration on solution pH over time was tested. 0.1 mEq/mL, 0.3 mEq/mL, and 1 mEq/mL sodium bicarbonate solutions were added to 100 mL polypropylene bags (manufacturer: Technoflex). The pH of each solution was measured at 0 days, 1 day, 2 days, 3 days, and 7 days. The results are shown in FIG. 2. The results surprisingly show that the pH of solutions comprising a higher concentration of sodium bicarbonate increases more slowly over time compared to the pH of solutions comprising lower concentrations of sodium bicarbonate.

### Example 9

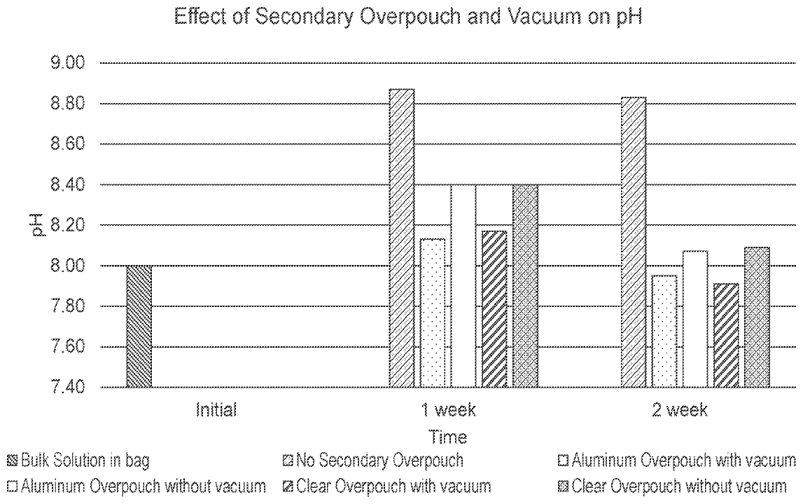

Effect of Secondary Overpouch and Vacuum on pH The effect of use of a secondary overpouch and/or vacuum on solution pH over time was tested. Sodium bicarbonate solutions were added to IV bags. Some bags were packaged without any secondary overpouch. Some bags were covered with a clear or aluminum overpouch. Other bags were covered with a clear or aluminum overpouch and the space between the IV bag and the overpouch was evacuated. The pH of the solutions in the IV bags was measured initially and 1 week and 2 weeks after preparation. The results are shown in FIG. 3. The results show that the pH of solutions stored in IV bags with overpouches increased less compared to the pH of solutions stored in IV bags without overpouches. The results further show that the pH of solutions in IV bags with overpouches that were evacuated increased less compared to the pH of solutions in IV bags with overpouches that were not evacuated. Surprisingly, the pH at two weeks after packaging decreased compared to one week after packaging in solutions that were stored in IV bags with an overpouch that was evacuated.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-82. (canceled)

83. A sterile, ready-to-use liquid pharmaceutical composition comprising:
sodium bicarbonate at a concentration of from about 0.05 mEq/mL to about 5 mEq/mL; and
carbon dioxide,
wherein the composition has an initial pH of 7.0 to 8.5,
wherein the composition is packaged in a container for intravenous use,
wherein the container is packaged in an overwrap, and
wherein the initial pH of the composition increases by less than 0.5 after storage for at least 6 months at 25° C.

84. The composition of claim 83, wherein the sodium bicarbonate is present at a concentration of from about 0.15 mEq/mL to about 1 mEq/mL.

85. The composition of claim 83, wherein the sodium bicarbonate is present at a concentration of about 0.1 mEq/mL.

86. The composition of claim 83, wherein the sodium bicarbonate is present at a concentration of about 0.25 mEq/mL.

87. The composition of claim 83, wherein the sodium bicarbonate is present at a concentration of about 0.5 mEq/mL.

88. The composition of claim 83, wherein the sodium bicarbonate is present at a concentration of about 0.75 mEq/mL.

89. The composition of claim 83, wherein the sodium bicarbonate is present at a concentration of about 0.8 mEq/mL.

90. The composition of claim 83, wherein the sodium bicarbonate is present at a concentration of about 1 mEq/mL.

91. The composition of claim 83, wherein the pH of the composition increases by less than 0.5 after storage for at least 12 months at 25° C.

92. The composition of claim 83, wherein the composition is preservative free.

93. The composition of claim 83, wherein the container comprises SiOₓ.

94. The composition of claim 83, wherein the container comprises an inner layer and an outer layer, and wherein the inner layer comprises SiOₓ.

95. The composition of claim 83, wherein a space between the container and the overwrap is evacuated.

96. The composition of claim 83, wherein the container is a single-dose container.

97. A method for alleviating metabolic acidosis in a subject comprising administering to the subject the composition of claim 83, wherein the subject has an arterial blood pH of less than 7.5.

98. The method of claim 97, wherein the metabolic acidosis is due to diabetic ketoacidosis, lactic acidosis, septic shock, intraoperative metabolic acidosis, or cardiac arrest.

99. The method of claim 97, wherein the composition is sterile and aseptic upon administration to the subject.

100. A method of replacing sodium bicarbonate in a subject comprising administering to the subject the composition of claim 83, wherein the subject suffers from sodium bicarbonate loss.

101. The method of claim 100, wherein the sodium bicarbonate loss is due to hyperchloremic acidosis resulting from diarrhea or renal proximal tubular acidosis.

102. The method of claim 100, wherein the composition is sterile and aseptic upon administration to the subject.

103. A method of treating kidney dysfunction in a subject, the method comprising administering to the subject the composition of claim 83.
